(19) United States
(12) Patent Application Publication
(10) Pub. No.: US 2026/0159872 A1
(43) Pub. Date: Jun. 11, 2026

(54) CENTRIFUGAL MICROFLUIDIC SYSTEMS AND METHODS FOR DNA METHYLATION SAMPLE PREPARATION

(71) Applicant: University of Virginia Patent Foundation, Charlottesville, VA (US)

(72) Inventors: Rachelle Ashley TURIELLO, Charlottesville, VA (US); Renna NOUWAIRI, Fisherville, VA (US); James P. LANDERS, Charlottesville, VA (US)

(21) Appl. No.: 19/150,118

(22) PCT Filed: Feb. 15, 2024
(86) PCT No.: PCT/US2024/016022

§ 371 Date: Jul. 22, 2025

Related U.S. Application Data
(60) Provisional application No. 63/445,977, filed on Feb. 15, 2023.

(51) Int. Cl. C12Q 1/6806; B01L 3/00

(52) U.S. Cl. CPC C12Q 1/6806; B01L 3/50273; B01L 3/502761; B01L 2200/0668; B01L 2200/16; B01L 2400/0409; B01L 2400/0633

(57) ABSTRACT

Various embodiments are provided for a centrifugal microfluidic device. In one example, the centrifugal microfluidic device includes a plurality of layers forming a body. The device can further include a plurality of sample preparation domains disposed in parallel within the body. The individual sample preparation domains include a lysis chamber configured to receive a sample from a swab and to receive a lysate solution. Additionally, the domains include a metering chamber configured to receive a lysate from the lysis chamber, where the metering chamber is connected to the lysis chamber via a first microfluidic channel having a laser valve. The domains can further include an overflow chamber configured to receive overflow of the lysate from the metering chamber. The domains can further include a bisulfite conversion portion of the sample preparation domain connected to the metering chamber via a second microfluidic channel.

### CROSS-REFERENCE TO RELATED APPLICATIONS

This application claims priority to, and the benefit of, co-pending U.S. provisional application entitled “CENTRIFUGAL MICROFLUIDIC SYSTEMS AND METHODS FOR DNA METHYLATION SAMPLE PREPARATION” having Ser. No. 63/445,977, filed Feb. 15, 2023, which is hereby incorporated by reference in its entirety.

### BACKGROUND

Deoxyribonucleic acid (DNA) methylation is a chemical modification that occurs at the C5 position of a cytosine reside. These modifications have been associated with cellular differentiation, gene expression, and eukaryotic aging. Epigenetic analysis to interrogate methylation status requires extensive sample preparation, including both DNA extraction to lyse and purify genetic material, and sodium bisulfite conversion to chemically modify genetic bases for downstream site identification. The preponderance of both DNA extraction and sodium bisulfite conversion methods requires some form of a silica solid phase to sequester genetic material and enable the multiple washing steps associated with DNA purification. Both processes are labor-intensive and time consuming, requiring multiple tube transfers and resulting in a tremendous loss of DNA template throughout the course of the sample preparation process

### SUMMARY

In accordance with the purpose(s) of this disclosure, as embodied and broadly described herein, the disclosure, in various aspects, relates to a microfluidic device that incorporates both upstream enzymatic lysis and sodium bisulfite conversion to provide a streamlined tool for epigenetic sample preparation and methods of use thereof. The solutions described herein combine various aspects of chemistry, biology, mechanical technologies, and microfluidic techniques to arrive at novel solutions for epigenetic sample preparation.

Aspects of the present disclosure provide for a rotationally-driven microfluidic device for integrated methylation sample preparation and methods of use thereof. Embodiments of the present disclosure include: a microfluidic device having a plurality of layers, wherein the layers include microfluidic chambers, chambers for swab inclusion, and microfluidic channels which can be ablated with a laser at various laser valves and channel closures.

A centrifugal microfluidic device is described herein, where the device has a plurality of layers which form a body. The device can have a plurality of sample preparation domains disposed in parallel within the body. Individual sample preparation domains includes a lysis chamber configured to receive a sample from a swab and to receive a lysis solution; a metering chamber configured to receive a lysate from the lysis chamber, where the metering chamber is connected to the lysis chamber via a first microfluidic channel having a laser valve; an overflow chamber configured to receive overflow of the lysate from the metering chamber; and a bisulfite conversion portion of the sample preparation domain connected to the metering chamber via a second microfluidic channel. According to various examples, the lysis solution includes at least a thermophilic neutral protease. In some embodiments, the lysis chamber of the device can further include a hydrophobic membrane configured to allow air to pass out of the lysis chamber. In some embodiments, the overflow chamber is accessible via pipette. In some embodiments, a laser valve is opened in the second microfluidic channel to allow the first fluid to flow from the metering chamber into the conversion chamber.

The bisulfite conversion portion of the device includes a conversion chamber which stores a conversion reagent. The conversion chamber can be configured to receive the lysate from the metering chamber and combine the lysate and the conversion reagent to produce a first fluid. The conversion chamber includes a hydrophobic membrane to vent gas from the conversion chamber. In some embodiments, the bisulfite conversion portion includes one or more reagent chambers storing one or more reagents, and a mixing chamber connected to the conversion chamber and the one or more reagent chambers. The mixing chamber can be configured to receive one or more reagents from the one or more reagent chambers and to receive the first fluid from the conversion chamber. The mixing chamber can further include a hydrophobic membrane to vent gas from the mixing chamber. Further, the bisulfite conversion portion includes an eluate collection chamber connected to the mixing chamber, where the eluate collection chamber can be configured to receive an eluate from the mixing chamber. In some embodiments, the bisulfite conversion portion includes one or more waste chambers connected to the mixing chamber, where the one or more waste chambers are configured to receive one or more reagent waste products from the mixing chamber.

The one or more reagent chambers includes a first wash buffer chamber, a desulphonation buffer chamber, a second wash buffer chamber, and an eluate buffer chamber, where the first and second wash buffer chambers contain ethanol or another wash buffer. According to various examples, the mixing chamber can further include a plurality of magnetically actuated beads and a bead binding buffer, where the plurality of magnetically actuated beads is configured to mix the first fluid with the one or more reagents to create the eluate and one or more reagent waste products. In some embodiments, the lysis chamber, the metering chamber, the conversion chamber, the mixing chamber and the eluate collection chamber are connected in series by a plurality of microfluidic channels, individual microfluidic channels of the plurality of microfluidic channels having at least one of a laser valve or a channel closure. In some embodiments, the eluate collection chamber is configured to receive the eluate from the mixing chamber through a microfluidic channel having an open laser valve. Further, in some embodiments, individual waste chambers of the one or more waste chambers can be connected in parallel by a microfluidic channel having one or more laser valves corresponding to the one or more waste chambers.

In some embodiments, at least one of the plurality of layers of the device is comprised of poly methyl methacrylate (PMMA) or poly(ethylene terephthalate) (PeT). In some embodiments, at least one of the plurality of layers is comprised of black poly(ethylene terephthalate) (bPeT). The plurality of layers can be adhered by at least one of a heat sensitive adhesive or a pressure sensitive adhesive.

A method of preparing nucleic acid for analysis using a centrifugal microfluidic device is described herein, where the method includes the steps of: receiving a sample of cells into a lysis chamber of the centrifugal microfluidic device; receiving a lysis solution into the lysis chamber; heating the lysis solution and the cells of the sample to form a lysate; flowing the lysate into a metering chamber and an overflow chamber using centrifugal force; flowing the lysate from the metering chamber to a conversion portion of the centrifugal microfluidic device; and performing sodium bisulfite conversion on the lysate. Moving the lysate from the lysis chamber to the metering chamber can further include laser ablating a first laser valve within a first microfluidic channel connecting the lysis chamber to the metering chamber, thereby allowing the lysate to pass through the first microfluidic channel. Performing sodium bisulfite conversion on the lysate can further include the steps of moving the lysate from the metering chamber to a conversion chamber using centrifugal force; combining the lysate with a conversion reagent within the conversion chamber to produce a first fluid; and moving the first fluid to a mixing chamber using centrifugal force. Next, the method includes mixing the first fluid with a plurality of binding beads; removing a first waste product from the mixing chamber to a first waste chamber; mixing one or more reagents with the binding beads to form resultant binding beads; and performing elution with the resultant binding beads to produce an eluate. In some embodiments, combining the lysate with the conversion reagent further includes heating the lysate and the conversion reagent in the conversion chamber. Additionally, mixing one or more reagents with the binding beads can further include the steps of: introducing a first wash buffer from a wash buffer chamber to the binding beads in the mixing chamber; mixing the binding beads with the first wash buffer; and removing a second waste product to a second waste chamber using centrifugal force. Next, the method includes introducing a desulphonation reagent from a desulphonation reagent chamber to the binding beads in the mixing chamber; mixing the binding beads with the desulphonation reagent; and removing a third waste product to a third waste chamber using centrifugal force. In some embodiments, the method includes introducing a second wash buffer from a second wash buffer chamber to the binding beads in the mixing chamber; mixing the binding beads with the second wash buffer to form resultant binding beads; removing a fourth waste product to a fourth waste chamber using centrifugal force; and heating the resultant binding beads in the mixing chamber. Finally, performing elution with the resultant binding beads further includes introducing an eluate buffer from an eluate buffer chamber to the resultant binding beads in the mixing chamber; mixing the resultant binding beads with the eluate buffer; heating the mixing chamber to form an eluate; and removing the eluate to an eluate collection chamber using centrifugal force. Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of a conventional DNA methylation interrogation sample preparation workflow according to various embodiments of the present invention. FIG. 1A is an example of a representative solid phase extraction strategy through a silica column. FIG. 1B is an example of a typical dynamic solid phase sodium bisulfite conversion workflow.

FIG. 2 is an alternative enzymatic lysis strategy according to various embodiments of the present invention. FIG. 2A shows an enzymatic protocol showing a one-step lysis strategy that would produce a lysate for introduction to the bisulfite conversion architecture (FIG. 2B) for microfluidic methylation sample preparation.

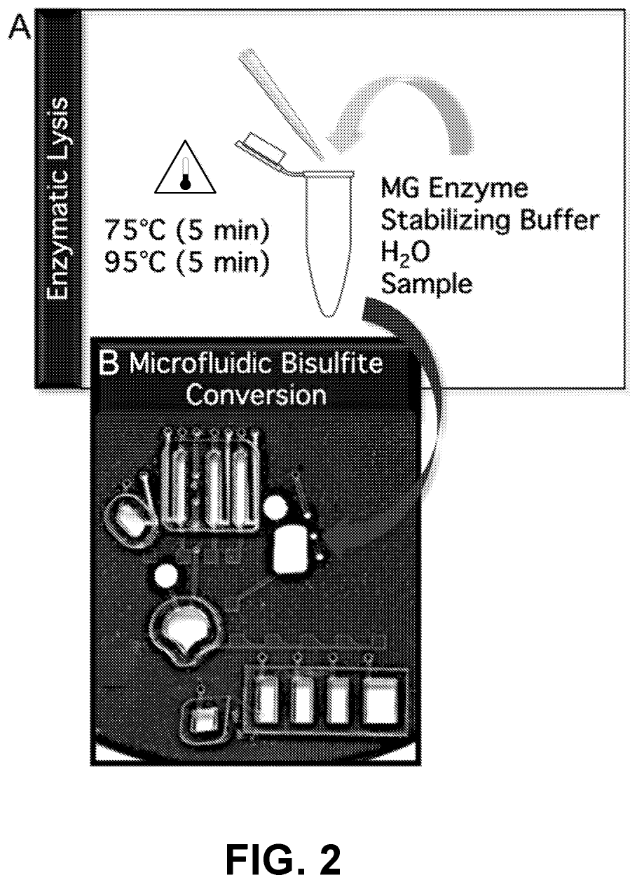

FIG. 3 is an example of architectural features of each polymeric layer belonging to the methylation sample preparation disc according to various embodiments of the present invention. The layered device of FIG. 3 includes five intermediate layers consisting of clear and black PeT and HSA. Fluidic chamber dimensions are increased for improved fluid capacity through the addition of 1.5 mm PMMA pieces on the top and bottom, which are covered in PeT to encapsulate reagents. Accessory pieces, including PeT coverlets, PMMA chambers for swab inclusion, and hydrophobic membranes are added last to enable direct-from-swab lysis and on-disc heated incubations.

FIG. 4 is an example of architectural details of the methylation sample preparation disc according to various embodiments of the present disclosure. FIG. 4A shows an aligned sample preparation disc showing five domains in parallel. FIG. 4B shows architectural features of one domain, highlighting the features associated with microfluidic lysis (top) and with sodium bisulfite conversion (bottom).

FIG. 5 shows an example of a fluidic dye study according to various embodiments of the present disclosure. One representative set of digital scans to show aqueous dye moving through one domain on the microfluidic disc including extraction, sulphonation and deamination (conversion 1), bead binding, wash #1, desulphonation (conversion 2), wash #2, and DNA elution (n=3).

FIG. 6 shows an example of extraction method comparison studies with K-562 cell lines according to various embodiments of the present disclosure. FIGS. 6A-B show a comparison of the enzymatic extraction method (MicroGEM) to the gold-standard solid-phase DNA extraction/purification method (Qiagen) in terms of DNA recovery (6A) and conversion efficiency (6B) (n=2, extraction & n=3, RT-PCR & HRM). FIGS. 6C-D show a comparison of conventional and shortened enzymatic extraction reaction intervals, including DNA recovery (6C) and conversion efficiency (6D) (n=3, extraction & n=3, RT-PCR & HRM).

FIG. 7 shows an example of an extraction method comparison studies with clinical blood samples according to various embodiments of the present disclosure. FIGS. 7A-B show a comparison of enzymatic extraction methods from MicroGEM, utilizing two proprietary buffer chemistries and the Qiagen gold-standard solid-phase extraction/purification chemistry (n=3 extraction, n=3 RT-PCR & HRM).

FIG. 8 shows an example of on-disc enzymatic lysis with clinical blood samples according to various embodiments of the present disclosure. FIG. 8A shows digital scans of the cellular lysis chamber with extraction cocktail pre-heating, post-heating, and post-centrifugation to pellet the heme. FIG. 8B shows RT-PCR results indicating higher recovery from chambers that “failed.” FIG. 8C shows corresponding HRM results from on-disc lysis. (n=3, RT-PCR & HRM).

FIG. 9 shows an example configuration of a microfluidic bisulfite conversion domain according to various embodiments of the present disclosure. FIG. 9A shows one domain with numbered chambers and positions of valves. FIG. 9B shows a key corresponding to the assigned chamber numbers and names. Valves are depicted as boxes, where those valves depicted in black correspond to ‘normally closed valves’ that are opened and those highlighted in gray correspond to ‘channel closures.’

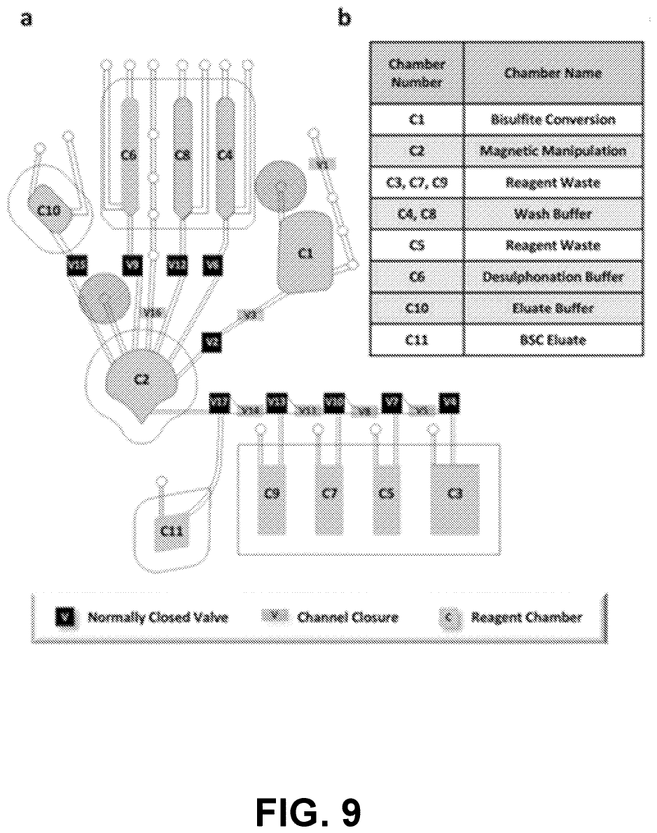

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, chemistry, biology, mechanical engineering, and microfluidic techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some embodiments, the term “about” includes traditional rounding according to significant figures of the numerical value. In addition, the phrase “about ‘x’ to ‘y’”, where ‘x’ and ‘y’ are numerical values, includes “about ‘x’ to about ‘y’”.

### Discussion

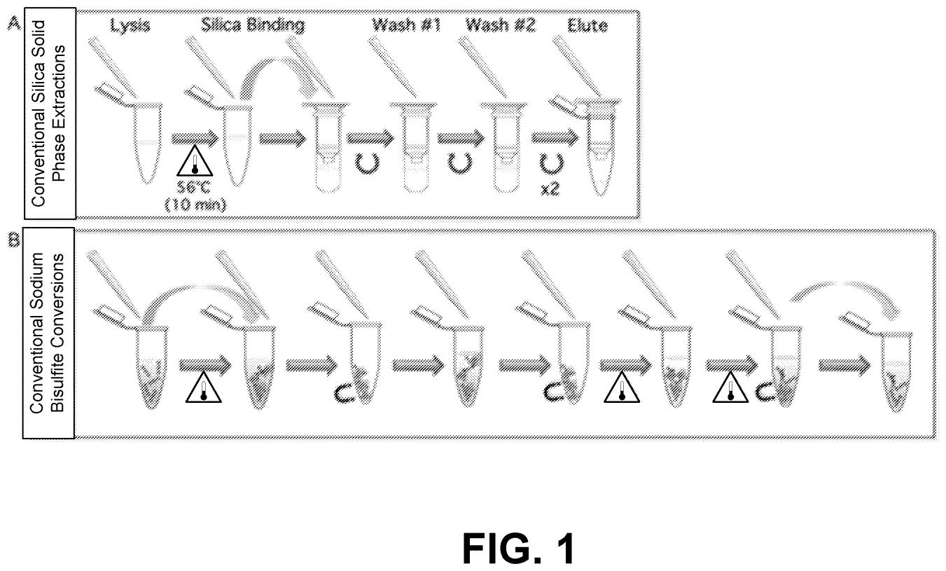

Disclosed are various approaches for DNA methylation sample preparation using centrifugal microfluidic systems. Previously, it has been demonstrated that the conversion portion of the epigenetic sample preparation process can be automated on a rotationally-driven microfluidic disc, whereby recovery and conversion efficiency are comparable to the gold-standard method. Fully automating this particular sample preparation workflow requires integration of upstream cellular lysis onto the device; however, incorporating another silica-based workflow would be difficult due to space constraints. Alternatively, an enzymatic method for cellular lysis sans purification could be easily integrated onto the disc. In this case, the multistep workflow characterized by a typical DNA extraction (FIG. 1A) would be distilled down to a single lysis step through the use of a thermophilic neutral protease, known as EA1 (MicroGEM International, PLC).

Accordingly, various embodiments of the present disclosure are directed to a microdevice that incorporates both upstream enzymatic lysis and sodium bisulfite conversion to provide a streamlined tool for epigenetic sample preparation. The present disclosure includes a rotationally-driven microfluidic platform for integrated methylation sample preparation, to include enzymatic cellular lysis using a neutral protease and dynamic solid phase sodium bisulfite conversion. This method streamlines an otherwise time-consuming and labor-intensive workflow, minimizing errors due to interoperability and open-tube handling steps. By leveraging the physics associated with microfluidics, namely the greatly increased surface-area-to-volume ratios of the chambers, incubation times are greatly reduced compared to the conventional methods. Thus, total analytical time is minimized through both automation with a corresponding mechatronic system (hardware) and via decreased incubation times for discrete processes completely within the disc. This alternative to the conventional method(s) is cost-effective and green, as it requires far fewer plastics (e.g., pipette tips, microcentrifuge tubes, etc.).

In principle, the centrifugal microfluidic system utilizes rotational forces as the single mechanism for fluid propulsion and will be particularly advantageous for both clinical and forensic applications for several reasons. First, a centrifugal device relies on rotationally-generated pressure to drive fluid flow, eliminating the need for bulky external hardware (e.g., syringe pumps, electronics, tubing) that hinder portability. Second, the fully closed nature of a centrifugal microfluidic system permits automation with minimal waste of both sample and reagents. Third, and finally, the forces controlling fluid movement through channels and into reaction and metering chambers for precise chemistries are easily controlled by adjusting rotational speed, which is easily automated. Therefore, the fully integrated, automated, and enclosed system described herein will minimize both variability and contamination risk through automation and integration to supplant the manual, open-tube steps required in traditional BSC methods.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

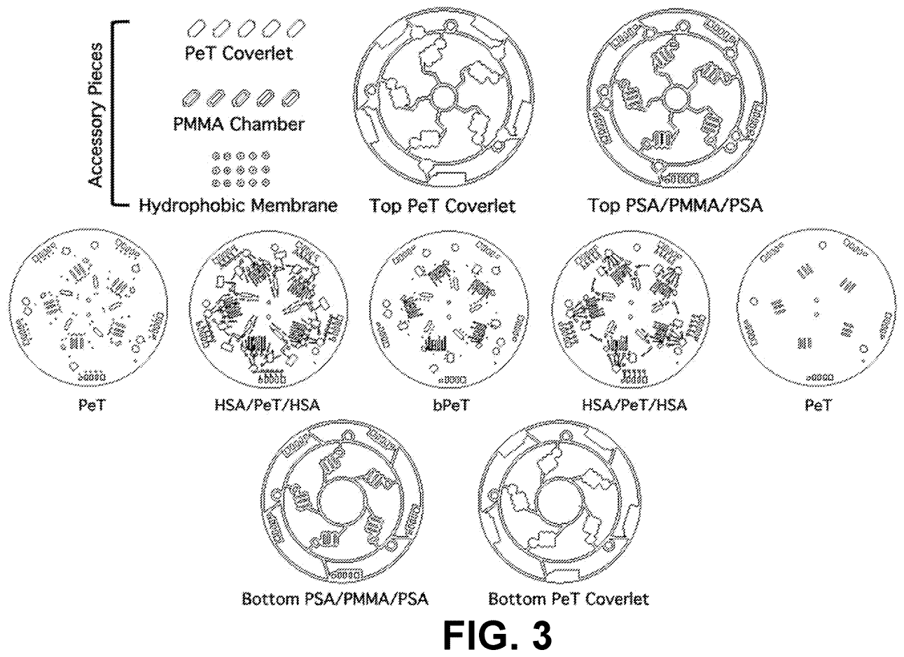

A centrifugal microfluidic device is described herein, where the device has a plurality of layers which form a body. Each layer includes various microstructures which are used to receive, store, transport, and conduct reactions on different fluids. In some embodiments, the device includes two top layers, five central layers, and two bottom layers (FIG. 3). In some embodiments, at least one of the plurality of layers of the device is comprised of poly methyl methacrylate (PMMA) or poly(ethylene terephthalate) (PeT). In some embodiments, at least one of the plurality of layers is comprised of black poly(ethylene terephthalate) (bPeT). For example, as shown in FIG. 3, the top layer is comprised of PeT, the second layer is comprised of PMMA, the fiver central layers are comprised of PeT with the central-most layer being comprised of bPeT, the second-to-bottom layer is comprised of PMMA, and the bottom layer is comprised of PeT. Various other combinations of PeT, bPeT, PMMA, and other similar materials are contemplated as well. The plurality of layers can be adhered by at least one of a heat sensitive adhesive or a pressure sensitive adhesive.

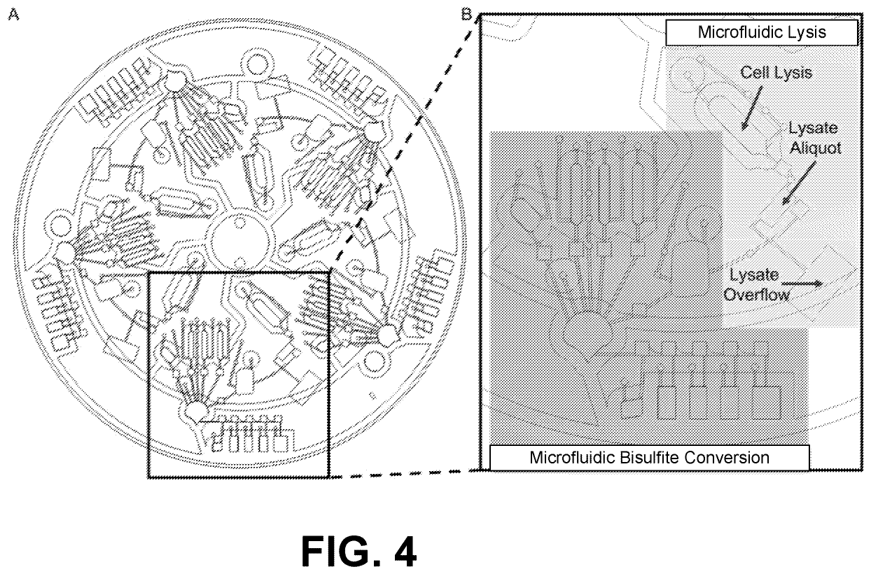

The device includes a plurality of sample preparation domains disposed in parallel within the body. Each sample preparation domain includes a microfluidic lysis portion of the device and a microfluidic bisulfite conversion portion of the device. As shown in the example of FIG. 4A, the device includes five separate sample preparation domains in parallel on the body of the device. Each domain spans the various layers of the device, with different chambers, channels, and valves on various respective layers. Fluid is moved through the device using controlled centrifugal force in combination with opening laser valves and closing channels.

Individual sample preparation domains include a lysis chamber configured to receive a sample from a swab and to receive a lysis solution. The lysis chamber of each domain spans through various layers of the device with at least one port for receiving the swab and one port for receiving the lysis solution. These respective ports are accessible through the top layer of the device. According to various examples, the lysis solution includes at least a thermophilic neutral protease, as well as other lysis solutions as can be appreciated. In some embodiments, the lysis chamber of the device further includes a hydrophobic membrane configured to allow air to pass out of the lysis chamber. This hydrophobic membrane serves as a vent to degas the fluid when it is heated during the lysis stage. Once the sample and the lysis solution have been received into the lysis chamber, the lysis chamber can be heated to produce a lysate.

A metering chamber is included in the device, where the metering chamber is configured to receive the lysate from the lysis chamber. In some embodiments, the metering chamber is configured to receive approximately two microliters of lysate fluid to be used in downstream preparation, while any excess lysate fluid flows into an overflow chamber. In some embodiments, the overflow chamber is accessible via pipette so overflow can be used to run simultaneous analyses. The metering chamber is connected to the lysis chamber via one microfluidic channel and, in some embodiments, to the overflow chamber via the same or another microfluidic channel. According to various examples, these microfluidic channels include laser valves, or sections of the microfluidic channels which indicate where to ablate with a laser to open the microfluidic channel such that the lysate can flow from the lysis chamber into the metering chamber, or from the metering chamber into the overflow chamber. Various similar laser valves are distributed throughout the microfluidic channels of the device, with at least one laser valve located between each major portion of the device.

The device further includes a bisulfite conversion portion of the sample preparation domain which is connected to the metering chamber via another microfluidic channel. The bisulfite conversion portion of the device includes a conversion chamber which stores a conversion reagent. The conversion chamber can be configured to receive the lysate from the metering chamber and combine the lysate and the conversion reagent to produce a first fluid. The first fluid includes ammonium bisulfite and DNA in varying amounts and configurations, as can be appreciated. In some embodiments, a laser valve in the microfluidic channel is opened, as described above, to allow the first fluid to flow from the metering chamber into the conversion chamber. The first fluid is heated in the conversion chamber. According to various examples, the conversion chamber also includes a hydrophobic membrane to vent gas from the conversion chamber during heating.

The mixing chamber is also connected to the conversion chamber. The mixing chamber is configured to receive one or more reagents from the respective reagent chambers and to receive the first fluid from the conversion chamber. In some examples, the mixing chamber further includes a hydrophobic membrane to vent gas from the mixing chamber during cycles which require heat. According to various examples, the mixing chamber further includes a plurality of magnetically actuated beads and a bead binding buffer, where the plurality of magnetically actuated beads is configured to mix the first fluid with the one or more reagents to create the eluate and one or more reagent waste products. The magnetically actuated beads, also referred to herein as binding beads, are mixed with the various reagents in the steps of bisulfite conversion.

In some embodiments, the bisulfite conversion portion includes one or more waste chambers connected to the mixing chamber, where the one or more waste chambers are configured to receive one or more waste products from the mixing chamber. At each step of the bisulfite process, waste products can be removed from the mixing chamber using centrifugal force to direct the waste out of the mixing chamber and into microfluidic channels connected to the waste chambers. Through laser valves and channel closures, or laser melting a portion of a microfluidic channel closed, the waste can be removed and sealed into a waste chamber. For example, the first fluid is mixed with a plurality of binding beads in the mixing chamber, to produce a first waste product. The first waste product is removed from the mixing chamber to a first waste chamber by ablating a laser valve in a microfluidic channel connecting the mixing chamber to the first waste chamber and using centrifugal force to drive the waste product into the waste chamber.

In some embodiments, the bisulfite conversion portion of the sample preparation domain includes one or more reagent chambers storing one or more reagents. In some embodiments, the reagent chambers include a first wash buffer chamber, a desulphonation buffer chamber, a second wash buffer chamber, and an eluate buffer chamber. In some embodiments, the first and second wash buffer chambers contain ethanol or another wash buffer. Each of the reagent chambers can be connected to a mixing chamber via a microfluidic channel including a laser valve. The laser valves to the reagent chambers are opened one at a time to allow for the various different cycles of bisulfite conversion to be conducted without contamination. For example, the first wash buffer channel is opened, and the first wash buffer is introduced to the mixing chamber where the binding beads are contained. The binding beads and the first wash buffer are mixed, forming a second waste product. Next, the second waste product is removed by opening a laser valve and using centrifugal force to remove the second waste product to a second waste chamber. Similarly, a desulphonation reagent is introduced by opening a laser valve connecting the mixing chamber to a desulphonation reagent chamber. The desulphonation reagent is mixed with the binding beads in the mixing chamber producing a third waste product. The third waste product can be removed to a third waste chamber by opening a laser valve and using centrifugal force. In some embodiments, a second wash buffer is introduced from a second wash buffer chamber to the binding beads in the mixing chamber. The binding beads are mixed with the second wash buffer to form resultant binding beads and a fourth waste product. The fourth waste product can be removed to a fourth waste chamber by opening laser valves and using centrifugal force. The resultant binding beads can be heated in the mixing chamber. In some embodiments, individual waste chambers of the one or more waste chambers can be connected in parallel by a single microfluidic channel having one or more laser valves corresponding to the one or more waste chambers.

Next, the bisulfite conversion portion further includes an eluate collection chamber connected to the mixing chamber, where the eluate collection chamber can be configured to receive an eluate from the mixing chamber. The eluate is formed by introducing an eluate buffer from an eluate buffer chamber to the resultant binding beads in the mixing chamber. The resultant binding beads are then mixed with the eluate buffer and heated to form an eluate. The eluate can be removed to the eluate collection chamber by opening laser valves and using centrifugal force. In some embodiments, the eluate collection chamber is configured to receive the eluate from the mixing chamber through a microfluidic channel having an open laser valve.

In some embodiments, the lysis chamber, the metering chamber, the conversion chamber, the mixing chamber and the eluate collection chamber are connected in series by a plurality of microfluidic channels, individual microfluidic channels of the plurality of microfluidic channels having at least one of a laser valve or a channel closure.

### Microdevice Fabrication

Rotationally-driven microdevice architecture was designed using AutoCAD software (Autodesk, Inc., Mill Valley, CA), then laser ablated into thermoplastic substrates using a CO2 laser (VLS 3.50, Universal Laser Systems, Scottsdale, AZ). The primary device consists of five primary poly(ethylene terephthalate) (PeT) layers (Film Source, Inc., Maryland Heights, MO) (FIG. 3). Layers 1 and 5 are considered capping layers and are composed solely of PeT. Layers 2 and 4 are the primary fluidic layers and are composed of PeT coated with a heat-sensitive adhesive (EL-7970-39, Adhesives Research, Inc., Glen Rock, PA). Layer 3, composed of black PeT (bPeT) (Lumirror X30, Toray Industries, Inc., Chuo-ku, Tokyo, Japan), acts as an intervening layer between the two primary fluidic layers to permit laser valving. Layers were aligned and heat-bonded using an office laminator (UltraLam 250B, Akiles Products, Inc., Mira Loma, CA) according to the “print, cut, laminate” method, described in detail elsewhere. Polymethyl methacrylate (PMMA) covers and accessory pieces (1.5 mm thickness, McMaster Carr, Elmurst, IL) are affixed to layers 1 and 5 by a pressure sensitive adhesive (PSA) transfer tape (MSX-7388, 3M, Saint Paul, MN), and capped with PeT to increase chamber depth and fluid capacity. Fluidic channels connecting microdevice chambers had a depth of approximately 100 μm and approximate widths between 400-500 μm. Each disc was designed with a view of multiplexing and includes domains capable of lysing and converting 5 samples in parallel. Each domain is capable of housing reagents including buffers, silica beads, and samples, features patches for laser valving, and hydrophobic membranes for gas exchange during heated incubations and for sample retrieval (FIG. 4).

### Characterization of the dSP-BSC Microdevice

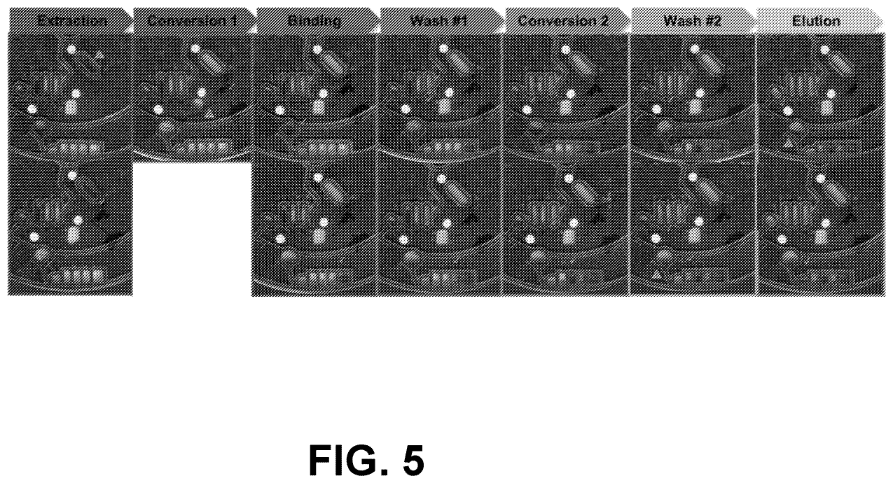

Early testing of the microfluidic architecture consisted of fluidic dye studies to ensure sequential unit operations were successful, including the sequestration of reagents during discrete processes and fluidic mixing via magnetic silica particles. FIG. 5 shows one representative dye study, whereby digital scans were taken of the disc following each fluidic step from extraction through the final DNA elution. Panels for each discrete step of the workflow include the chemical step (upper panel) and the requisite removal of waste to a corresponding chamber at the periphery of the disc (bottom panel).

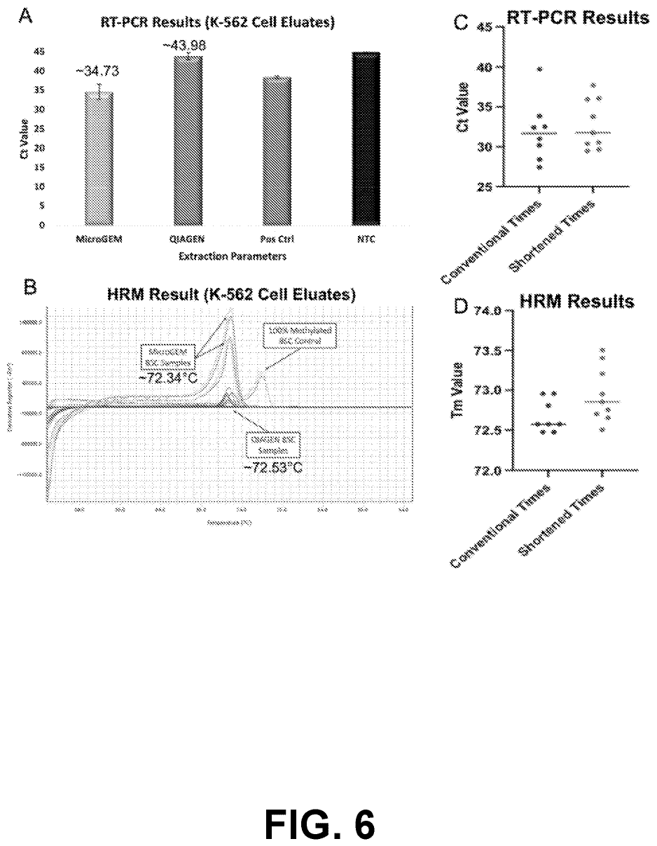

The decision to utilize an enzymatic method for cellular lysis sans purification required some proof-of-concept studies to ensure the extraction method was compatible with downstream sodium bisulfite conversion and it performed comparably to a gold-standard method for extraction in terms of DNA recovery and bisulfite conversion efficiency. For a preliminary study, K-562 cell line samples at equivalent concentrations (125 k) were extracted in parallel by a column-based, solid-phase Qiagen chemistry (DNeasy Blood and Tissue Kit, Qiagen) and the MicroGEM enzymatic method (prepGEM Universal, MicroGEM). Relative DNA recovery was evaluated via RT-PCR, whereby resultant Cₜ values were compared; because these values are representative of starting concentration, it follows that samples prepared with optimal sample preparation conditions for DNA preservation would produce more rapid amplification (e.g, lower Cₜ values). Here, the ZymoTaq™ DNA Polymerase chemistry was used, as it was specifically designed for the amplification of bisulfite-treated DNA; however, the protocol was modified for reagent conservation and to enable real-time detection by utilizing only half reactions and adding an intercalating Syto 9 dye, respectively. Likewise, relative conversion efficiency was demonstrated with HRM analysis, whereby the Tm of DNA samples post sample preparation processing were compared to show equivalent conversion of unmethylated cytosines in the template. RT-PCR results indicate that recovery is greatly enhanced with the enzymatic method compared to the gold-standard counterpart; FIG. 6A shows Cₜ values originating from the MicroGEM method average ˜34.73, whereas samples amplified following Qiagen preparation had average Cₜ values of ˜43.98. With regard to conversion efficiency, fluorescent signal was much lower for samples prepared with the Qiagen method (FIG. 6B), but no statistical differences were observed between Tm values (p-value=0.15, α=0.05). It was concluded that while both methods are compatible with the downstream BSC method, the enzymatic method outperformed the gold-standard method in terms of DNA recovery and was comparable in resulting conversion efficiency. Moving forward, the inventors sought to decrease the incubation times associated with the enzymatic extraction method prior to incorporating it onto the disc, mitigating the potential for extended periods of thermal expansion on the disc and thus of heating-related fluid loss. The conventional incubation intervals consist of two steps spanning five minutes each at 75° C. and 95° C.; the inventors sought to decrease these intervals to one minute each, hypothesizing similar recovery given that the volume of the reaction was reduced by half and the SA:V ratio increased compared to the in-tube method. K-562 cell line samples at equivalent concentrations (125 k) were again run in parallel with either conventional incubation times or shortened incubation times. Results demonstrated no statistical differences between resultant DNA recovery (p-value=0.61, α=0.05) (FIG. 6C) or conversion efficiency (p-value=0.070, α=0.05) (FIG. 6D).

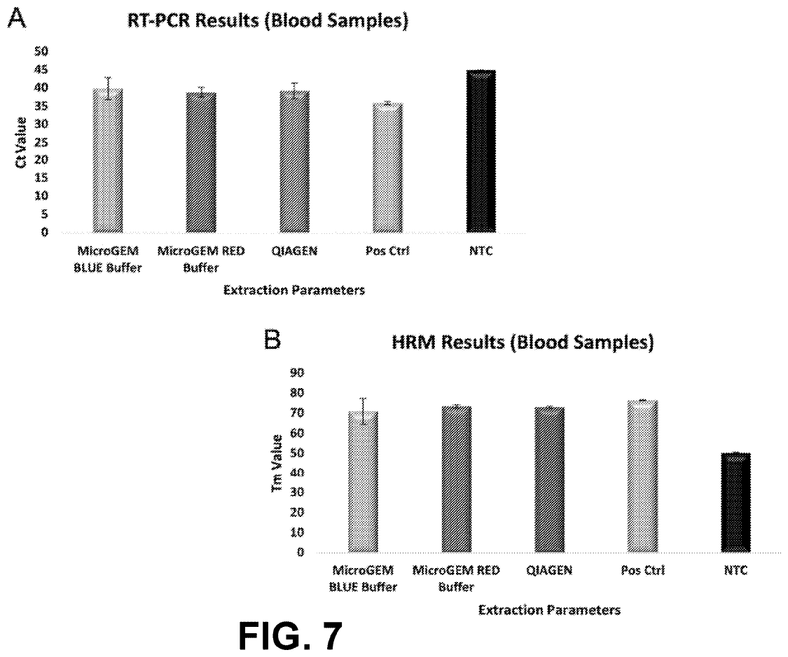

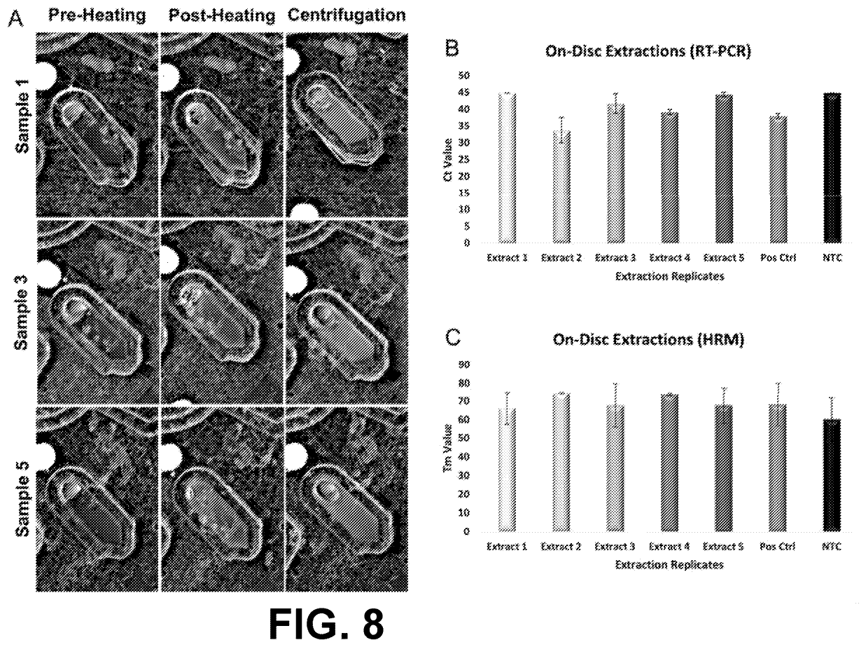

While the analysis of K-562 cell lines can provide some insight into performance of the chemistry, relevant samples will ultimately consist of body fluids containing a much more complex matrix. Thus, the next step was to verify downstream BSC compatibility, and comparable recovery and conversion efficiency from clinical blood samples obtained from UVA hospital. For proof-of-concept, aliquots from one clinical blood sample were extracted via one of two MicroGEM methods and the gold-standard solid-phase method described previously (Qiagen). The two enzymatic strategies employ the same enzyme in the same concentration for cellular lysis, however, they utilize two distinguished, proprietary buffer systems. Comparing both the RT-PCR results (FIG. 7A) and the HRM results (FIG. 7B), there were no statistical differences observed between eluates produced by either method (p-values=0.66 and 0.40, respectively, α=0.05) indicating no difference between resultant DNA recovery or conversion efficiency. Moving forward, the MicroGEM BLUE Buffer chemistry was tested on-disc (n=5), with two of five chambers tested experiencing device failure (e.g., leaking from the chamber onto the surface of the microdevice during heating). FIG. 8A shows digital scans taken from three “successfully heated replicates” pre-heating, post-heating, and post-centrifugation. Supernatant was subsequently removed from the chamber via pipette, bisulfite converted using conventional parameters, amplified via RT-PCR, and melted via HRM, as before. Eluates from chambers 2 and 4 were similarly removed from the disc and tested as well to compare recovery. Interestingly, RT-PCR results demonstrated that Cₜ values indicated higher recovery from those eluates that had not remained in the chamber for the final centrifugation step (FIG. 8B), indicating the heme pelleting step may be unnecessary and even detrimental to the overall success of the assay. HRM results were largely inconsistent (FIG. 8C) and will require further testing via microchip electrophoresis or pyrosequencing to fully understand.

Testing is ongoing, with future work focused on continued characterization of the enzymatic lysis strategy on-disc and integration with on-disc sodium bisulfite conversion downstream. Continued validation of the integrated sample preparation method is planned to demonstrate sensitivity, reproducibility, and robusticity of the system. Beyond RT-PCR and HRM, isolation and conversion of one complete template will be verified via microchip electrophoresis and conversion efficiency will be more thoroughly evaluated with a pyrosequencing assay.

### REFERENCES

The devices, systems, apparatuses, modules, compositions, articles of manufacture, materials, computer program products, non-transitory computer readable medium, and methods of various embodiments of the invention disclosed herein may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
A. International Patent Application Serial No. PCT/US2022/077977, entitled “A MICROFLUIDIC STRUCURE FOR DIFFERENTIAL EXTRACTION”, filed Oct. 12, 2022.
B. International Patent Application Serial No. PCT/US2022/076372, entitled “ACOUSTIC TRAPPING MICROCHANNEL RESONANCE DETECTION AND CONTROL”, filed Sep. 13, 2022.
C. U.S. Utility patent application Ser. No. 17/512,405, entitled “STORAGE OF CORROSIVE MATERIALS ON A FIBER-BASED FLUIDIC DEVICE AND RELATED METHODS THEREOF”, filed Oct. 27, 2021; Publication No. US 2022-0126293 A1, Apr. 28, 2022.
D. U.S. Utility patent application Ser. No. 17/447,015, entitled “DEVICES AND METHODS FOR EXTRACTION, SEPARATION AND THERMOCYCLING”, filed 15 Sep. 7, 2021; Publication No. US 2022-0097042 A1, Mar. 31, 2022.
E. U.S. Utility patent application Ser. No. 15/768,115, entitled “DEVICES AND METHODS FOR EXTRACTION, SEPARATION AND THERMOCYCLING”, filed Apr. 13, 2018; U.S. Pat. No. 11,135,583, issued Oct. 5, 2021.
F. International Patent Application Serial No. PCT/US2016/056906, entitled “DEVICES AND METHODS FOR EXTRACTION, SEPARATION AND THERMOCYCLING”, filed Oct. 13, 2016; Publication No. WO 2017/066485, Apr. 20, 2017.
G. U.S. Utility patent application Serial No. 16/484,142, entitled “MICRODEVICE FOR DIFFERENTIAL SEPARATION, PURIFICATION AND AMPLIFICATION OF FORENSIC SAMPLES”, filed Aug. 7, 2019; Publication No. US-2020-0023366-A1, Jan. 23, 2020.
H. International Patent Application Serial No. PCT/US2018/017213, entitled “MICRODEVICE FOR DIFFERENTIAL SEPARATION, PURIFICATION AND AMPLIFICATION OF FORENSIC SAMPLES”, filed Feb. 7, 2018; Publication No. WO 2018/148271, Aug. 16, 2018.
I. U.S. Utility patent application Ser. No. 16/225,527, entitled “DEVICES, SYSTEMS, AND METHODS FOR DETECTING SUBSTANCES”, filed Dec. 19, 2018; U.S. Pat. No. 11,207,677, issued Dec. 28, 2021.
J. U.S. Utility patent application Ser. No. 16/039,184, entitled “METHOD FOR DETECTING NUCLEIC ACIDS BASED ON AGGREGATE FORMATION”, filed Jul. 18, 2018.
K. U.S. Utility patent application Ser. No. 13/699,983, entitled “METHOD FOR DETECTING NUCLEIC ACIDS BASED ON AGGREGATE FORMATION”, filed Nov. 26, 2012; Publication No. 2013/0203045, Aug. 8, 2013.
L. International Patent Application Serial No. PCT/US2011/038166, entitled “METHOD FOR DETECTING NUCLEIC ACIDS BASED ON AGGREGATE FORMATION”, filed May 26, 2011; Publication No. WO 2011/150226, Dec. 1, 2011.
M. U.S. Utility patent application Ser. No. 15/770,413, entitled “DEVICES, SYSTEMS AND METHODS FOR SAMPLE DETECTION”, filed Apr. 23, 2018; Publication No. US-2019-0054468-A1, Feb. 21, 2019.
N. International Patent Application Serial No. PCT/US2016/058263, entitled “DEVICES, SYSTEMS AND METHODS FOR SAMPLE DETECTION”, filed Oct. 21, 2016; Publication No. WO 2017/070571, Apr. 27, 2017.
O. U.S. Utility patent application Ser. No. 15/770,035, entitled “SYSTEMS, DEVICES AND METHODS FOR ANALYZING AND IDENTIFYING SUBSTANCES”, filed Apr. 20, 2018; Publication No. US-2018-0313765-A1, Nov. 1, 2018.
P. International Patent Application Serial No. PCT/US2016/058304, entitled “SYSTEMS, DEVICES AND METHODS FOR ANALYZING AND IDENTIFYING SUBSTANCES”, filed Oct. 21, 2016; Publication No. WO 2017/070607, Apr. 27, 2017.
Q. U.S. Utility patent application Ser. No. 14/650,743, entitled “FREQUENCY-BASED FILTERING OF MECHANICAL ACTUATION USING FLUIDIC DEVICE”, filed Jun. 9, 2015; U.S. Pat. No. 9,987,576, issued Jun. 5, 2018.
R. International Patent Application Serial No. PCT/US2013/074152, entitled “FREQUENCY-BASED FILTERING OF MECHANICAL ACTUATION USING FLUIDIC DEVICE”, filed Dec. 10, 2013; Publication No. WO 2014/093360, Jun. 19, 2014.
S. U.S. Utility patent application Ser. No. 14/503,955, entitled “MICROFLUIDIC VALVE SYSTEMS”, filed Oct. 1, 2014; Publication No. 2015/0093838, Apr. 2, 2015.
T. U.S. Utility patent application Ser. No. 13/849,691, entitled “Electrostatic Actuation for Management of Flow in Micro Total Analysis Systems and Related Method Thereof”, filed Mar. 25, 2013.
U. U.S. Utility patent application Ser. No. 13/294,908, entitled “Electrostatic Actuation for Management of Flow in Micro-Total Analysis Systems and Related Method Thereof”, filed Nov. 11, 2011; U.S. Pat. No. 8,403,294, issued Mar. 26, 2013.
V. U.S. Utility patent application Ser. No. 11/665,326, entitled “Electrostatic Actuation for Management of Flow in Micro Total Analysis Systems and Related Method Thereof”, filed Apr. 13, 2007; U.S. Pat. No. 8,056,881, issued Nov. 15, 2011.
W. International Patent Application Serial No. PCT/US2005/036675, entitled “Electrostatic Actuation for Management of Flow in Micro Total Analysis Systems and Related Method Thereof”, filed Oct. 13, 2005; Publication No. WO06044458, Apr. 27, 2006.
X. U.S. Utility patent application Ser. No. 13/474,420, entitled “Passive Components for Micro-Fluidic Flow Profile Shaping and Related Method Thereof”, filed May 17, 2012; U.S. Pat. No. 9,050,596, issued Jun. 9, 2015.
Y. U.S. Utility patent application Ser. No. 12/064,557, entitled “Passive Components for Micro-Fluidic Flow Profile Shaping and Related Method Thereof”, filed Feb. 22, 2008; U.S. Pat. No. 8,220,493, issued Jul. 17, 2012.
Z. International Patent Application Serial No. PCT/US2006/032717, entitled “Passive Components for Micro-Fluidic Flow Profile Shaping and Related Method Thereof”, filed Aug. 23, 2006; Publication No. WO 2007/024829, Mar. 1, 2007.
AA. International Patent Application Serial No. PCT/US2012/036105, entitled “METHOD AND SYSTEM FOR HIGH THROUGHPUT OPTICAL AND LABEL FREE DETECTION OF ANALYTES”, filed May 2, 2012; Publication No. WO 2012/151268, Nov. 8, 2012.
BB. International Patent Application Serial No. PCT/US2012/036139, entitled “METHOD AND SYSTEM TO DETECT AGGREGATE FORMATION ON A Substrate”, filed May 2, 2012; Publication No. WO 2012/151289, Nov. 8, 2012.
CC. U.S. Utility patent application Ser. No. 13/505,406, entitled “VERSATILE, VISIBLE METHOD FOR DETECTING POLYMERIC ANALYTES”, filed May 1, 2012; Publication No. 2013/0084565, Apr. 4, 2013.
DD. U.S. Utility patent application Ser. No. 13/116,659, entitled “METHOD FOR DETECTING NUCLEATED CELLS”, filed May 26, 2011; Publication No. 2012/0149587, Jun. 14, 2012.
EE. International Patent Application Serial No. PCT/US2010/002883, entitled “VERSATILE, VISIBLE METHOD FOR DETECTING POLYMERIC ANALYTES”, filed Nov. 3, 2010; Publication No. WO 2011/056215, May 12, 2011.
FF. U.S. Utility patent application Ser. No. 12/879,810, entitled “DETECTION OF POLYMERIC ANALYTES”, filed Sep. 10, 2010; U.S. Pat. No. 10,656,146, issued May 19, 2020.
GG. International Patent Application Serial No. PCT/US2009/036983, entitled “DETECTION OF POLYMERIC ANALYTES”, filed Mar. 12, 2009; Publication No. WO 2009/114709, Sep. 17, 2009.
HH. U.S. Utility patent application Ser. No. 12/892,618, entitled “DNA EXTRACTION USING A PHOTO-POLYMERIZED MONOLITH IN A CAPILLARY”, filed Sep. 28, 2010; U.S. Pat. No. 9,044,735, issued June 2, 2015.
II. U.S. Utility patent application Ser. No. 11/885,181, entitled “Grafted Photo-Polymerized Monolithic Column”, filed Aug. 28, 2007; U.S. Pat. No. 7,815,802, issued Oct. 19, 2010.
JJ. International Patent Application Serial No. PCT/US2006/006845, entitled “DNA Extraction Using a Photo-Polymerized Monolith in a Capillary”, filed Feb. 28, 2006; Publication No. WO06093865, Sep. 8, 2006.
KK. International Patent Application Serial No. PCT/US2010/026791, entitled “MULTIPLE-SAMPLE MICROFLUIDIC CHIP FOR DNA ANALYSIS”, filed Mar. 10, 2010; Publication No. WO 2010/141139, Dec. 9, 2010.
LL. International Patent Application Serial No. PCT/US2010/026801, entitled “Optical Approach for Microfluidic DNA Electrophoresis Detection”, filed Mar. 10, 2010; Publication No. WO 2010/141140, Dec. 9, 2010.
MM. U.S. Utility patent application Ser. No. 12/090,233, entitled “Integrated Microfluidic Analysis Systems”, filed Apr. 14, 2008; U.S. Pat. No. 8,916,375, issued Dec. 23, 2014.
NN. International Patent Application Serial No. PCT/US2006/039809, entitled “Integrated Microfluidic Analysis Systems”, filed Oct. 12, 2006; Publication No. WO 2007/047336, Apr. 26, 2007.
OO. U.S. Utility patent application Ser. No. 12/089,320, entitled “Microchip-based Acoustic Trapping or Capture of Cells for Forensic Analysis and Related Method Thereof”, filed Apr. 4, 2008; Publication No. 2011/0033922, Feb. 10, 2011.
PP. International Patent Application Serial No. PCT/US2006/038943, entitled “Microchipbased Acoustic Trapping or Capture of Cells for Forensic Analysis and Related Method Thereof”, filed Oct. 4, 2006; Publication No. WO 2007/041671, Apr. 12, 2007.
QQ. U.S. Utility patent application Ser. No. 11/989,794, entitled “Microdevices for Chemical Sensing and Chemical Actuation”, filed Jan. 31, 2008; U.S. Pat. No. 8,343,755, issued Jan. 1, 2013.
RR. International Patent Application Serial No. PCT/US2006/030127, entitled “Microdevices for Chemical Sensing and Chemical Actuation”, filed Aug. 1, 2006; Publication No. WO 2007/030240, Mar. 15, 2007.
SS. U.S. Utility patent application Ser. No. 11/793,428, entitled “Use of Microwaves for Thermal and Non-Thermal Applications in Micro and Nanoscale Devices”, filed Jan. 7, 2008; Publication No. 2008/0277387, Nov. 13, 2008.
TT. International Patent Application Serial No. PCT/US2005/046756, entitled “The Use of Microwaves for Thermal and Non-Thermal Applications in Micro and Nanoscale Devices”, filed Dec. 22, 2005; Publication No. WO06069305, Jun. 29, 2006.
UU. International Patent Application Serial No. PCT/US2007/088662, entitled “Non-Contact Thermal Control of Small Volume and Related Apparatus Thereof”, filed Dec. 21, 2007; Publication No. WO 2008/080106, Jul. 3, 2008.
VV. International Patent Application Serial No. PCT/US2007/083964, entitled “DNA Purification in a Multi-Stage, Multi-Phase Microchip”, filed Nov. 7, 2007; Publication No. WO 2008/058204, May 15, 2008.
WW. U.S. Utility patent application Ser. No. 11/884,351, entitled “Nucleic Acid Isolation Methods and Materials and Devices Thereof”, filed Aug. 15, 2007; Publication No. 2009/0215124, Aug. 27, 2009.
XX. International Patent Application Serial No. PCT/US2006/005241, entitled “Nucleic Acid Isolation Methods and Materials and Devices Thereof”, filed Feb. 15, 2006; Publication No. WO06088907, Aug. 24, 2006.
YY. U.S. Utility patent application Ser. No. 11/664,297, entitled “Localized Control of Thermal Properties on Microdevices and Applications Thereof”, filed Mar. 19, 2007; Publication No. 2008/0193961, Aug. 14, 2008.
ZZ. International Patent Application Serial No. US2005/034674, entitled “Localized Control of Thermal Properties on Microdevices and Applications Thereof”, filed Sep. 29, 2005; Publication No. WO06039293, Apr. 13, 2006.
AAA. U.S. Utility patent application Ser. No. 10/535,926, entitled “Isolation of Sperm Cells from Other Biological Materials Using Microfabricated Devices and Related Methods Thereof”, filed May 23, 2005; Publication No.-060144707, Jul. 6, 2006.
BBB. International Patent Application Serial No. US2003/037205, entitled “Isolation of Sperm Cells from Other Biological Materials Using Microfabricated Devices and Related Methods Thereof”, filed Nov. 20, 2003; Publication No WO04046712, Jun. 3, 2004.
CCC. U.S. Utility patent application Ser. No. 10/530,728, entitled “Methods and Systems for Multiplexing IR-Mediated Heating on a Microchip”, filed Apr. 8, 2005; Publication No. 05-0287661, Dec. 29, 2005.
DDD. International Patent Application Serial No. US2003/031806, entitled “Methods and Systems for Multiplexing IR Mediated Heating on a Microchip”, filed Oct. 8, 2003; Publication No. WO04033099, Apr. 22, 2004.
EEE. International Patent Application Serial No. US2005/005490, entitled “Method and System for Eluting Cells”, filed Feb. 22, 2005; Publication No. WO05079531, Sep. 1, 2005.
FFF. U.S. Utility patent application Ser. No. 10/520,763, entitled “Hybrid Polymers for Functional Tuning of Microfluidic Device Surfaces”, filed Jan. 10, 2005; U.S. Pat. No. 7,381,471, issued Jun. 3, 2008.
GGG. International Patent Application Serial No. US2003/022162, entitled “Hybrid Polymers for Functional Tuning of Microfluidic Device Surfaces”, filed Jul. 15, 2003; Publication No. 04/007582, Jan. 22, 2004.
HHH. U.S. Utility patent application Ser. No. 10/517,980, entitled “Apparatus and Method for Purification of Nucleic Acids”, filed Dec. 13, 2004; U.S. Pat. No. 7,534,623, issued May 19, 2009.
III. International Patent Application Serial No. US2003/018403, entitled “Apparatus and Method for Purification of Nucleic Acids”, filed Jun. 11, 2003; Publication No. WO03104774, Dec. 18, 2003.
JJJ. International Patent Application Serial No. US2004/001276, entitled “METHOD FOR MICROCHIP AND CAPILLARY DETECTION OF PROTEINS IN THE SUB-UG/ML RANGE”, filed Jan. 20, 2004; Publication No. WO 2005/033687, Apr. 14, 2005.
KKK. International Patent Application Serial No. US2003/029249, entitled “Remote Temperature Sensing of Small Volume and Related Apparatus Thereof”, filed Sep. 17, 2003; Publication No. WO04027361, Apr. 1, 2004.
LLL. U.S. Utility patent application Ser. No. 10/664,064, entitled “Remote Temperature Sensing of Small Volume and Related Apparatus Thereof”, filed Sep. 17, 2003; Publication No.-0131504-, Jul. 8, 2004.
MMM. U.S. Utility patent application Ser. No. 10/432,141, entitled “Method for Orthogonal Analyte Stacking/Injection Systems in Electrophoresis”, filed May 16, 2003; U.S. Pat. No. 7,223,325, issued May 29, 2007.
NNN. International Patent Application Serial No. US01/43259, entitled “METHOD FOR ORTHOGONAL ANALYTE STACKING/INJECTION SYSTEMS IN ELECTROPHORESIS”, filed Nov. 19, 2001; Publication No. WO 02/48673, Jun. 20, 2002.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A centrifugal microfluidic device, comprising:
a plurality of layers forming a body;
a plurality of sample preparation domains disposed in parallel within the body, individual sample preparation domains comprising:
a lysis chamber configured to receive a sample from a swab and to receive a lysis solution;
a metering chamber configured to receive a lysate from the lysis chamber,
the metering chamber connected to the lysis chamber via a first microfluidic channel having a laser valve;
an overflow chamber configured to receive overflow of the lysate from the metering chamber; and
a bisulfite conversion portion of the sample preparation domain connected to the metering chamber via a second microfluidic channel.

2. The centrifugal microfluidic device of claim 1, wherein the lysis chamber further comprises a hydrophobic membrane configured to allow air to pass out of the lysis chamber.

3. The centrifugal microfluidic device of claim 1, wherein the bisulfite conversion portion further comprises:
a conversion chamber storing a conversion reagent, the conversion chamber configured to receive the lysate from the metering chamber and combine the lysate and the conversion reagent to produce a first fluid;
one or more reagent chambers storing one or more reagents;
a mixing chamber connected to the conversion chamber and the one or more reagent chambers and configured to receive one or more reagents from the one or more reagent chambers and to receive the first fluid from the conversion chamber;
an eluate collection chamber connected to the mixing chamber, the eluate collection chamber configured to receive an eluate from the mixing chamber; and
one or more waste chambers connected to the mixing chamber, the one or more waste chambers configured to receive one or more reagent waste products from the mixing chamber.

4. The centrifugal microfluidic device of claim 3, wherein the one or more reagent chambers include a first wash buffer chamber, a second wash buffer chamber, a desulphonation buffer chamber, and an eluate buffer chamber.

5. The centrifugal microfluidic device of claim 3, wherein a laser valve is opened in the second microfluidic channel to allow the first fluid to flow from the metering chamber into the conversion chamber.

6. The centrifugal microfluidic device of claim 3, wherein the mixing chamber further comprises a plurality of magnetically actuated beads and a bead binding buffer, the plurality of magnetically actuated beads configured to mix the first fluid with the one or more reagents to create the eluate and one or more reagent waste products.

7. The centrifugal microfluidic device of claim 3, wherein the lysis chamber, the metering chamber, the conversion chamber, the mixing chamber and the eluate collection chamber are connected in series by a plurality of microfluidic channels, individual microfluidic channels of the plurality of microfluidic channels having at least one of a laser valve or a channel closure.

8. The centrifugal microfluidic device of claim 3, wherein the eluate collection chamber is configured to receive the eluate from the mixing chamber through a microfluidic channel having an open laser valve.

9. The centrifugal microfluidic device of claim 3, wherein individual waste chambers of the one or more waste chambers are connected in parallel by a microfluidic channel having one or more laser valves corresponding to the one or more waste chambers.

10. The centrifugal microfluidic device of claim 1, wherein the overflow chamber is accessible via pipette.

11. The centrifugal microfluidic device of claim 1, wherein at least one of the plurality of layers is comprised of poly methyl methacrylate (PMMA) or poly(ethylene terephthalate) (PeT).

12. The centrifugal microfluidic device of claim 1, wherein at least one of the plurality of layers is comprised of black poly(ethylene terephthalate) (bPeT).

13. The centrifugal microfluidic device of claim 1, wherein the plurality of layers are adhered by at least one of a heat sensitive adhesive or a pressure sensitive adhesive.

14. The centrifugal microfluidic device of claim 1, wherein the lysis solution comprises at least a thermophilic neutral protease.

15. A method of preparing nucleic acid for analysis using a centrifugal microfluidic device, the method comprising:
receiving a sample of cells into a lysis chamber of the centrifugal microfluidic device;
receiving a lysis solution into the lysis chamber;
heating the lysis solution and the cells of the sample to form a lysate;
flowing the lysate into a metering chamber and an overflow chamber using centrifugal force;
flowing the lysate from the metering chamber to a conversion portion of the centrifugal microfluidic device; and
performing sodium bisulfite conversion on the lysate.

16. The method of claim 15, wherein moving the lysate from the lysis chamber to the metering chamber further comprises laser ablating a first laser valve within a first microfluidic channel connecting the lysis chamber to the metering chamber, thereby allowing the lysate to pass through the first microfluidic channel.

17. The method of claim 15, wherein performing sodium bisulfite conversion on the lysate further comprises:
moving the lysate from the metering chamber to a conversion chamber using centrifugal force;
combining the lysate with a conversion reagent within the conversion chamber to produce a first fluid;
moving the first fluid to a mixing chamber using centrifugal force;
mixing the first fluid with a plurality of binding beads;
removing a first waste product from the mixing chamber to a first waste chamber;
mixing one or more reagents with the binding beads to form resultant binding beads; and
performing elution with the resultant binding beads to produce an eluate.

18. The method of claim 17, wherein combining the lysate with the conversion reagent further comprises heating the lysate and the conversion reagent in the conversion chamber.

19. The method of claim 17, wherein mixing one or more reagents with the binding beads further comprises:
introducing a first wash buffer from a wash buffer chamber to the binding beads in the mixing chamber;
mixing the binding beads with the first wash buffer;
removing a second waste product to a second waste chamber using centrifugal force;
introducing a desulphonation reagent from a desulphonation reagent chamber to the binding beads in the mixing chamber;
mixing the binding beads with the desulphonation reagent;
removing a third waste product to a third waste chamber using centrifugal force;
introducing a second wash buffer from a second wash buffer chamber to the binding beads in the mixing chamber;
mixing the binding beads with the second wash buffer to form resultant binding beads;
removing a fourth waste product to a fourth waste chamber using centrifugal force; and
heating the resultant binding beads in the mixing chamber.

20. The method of claim 17, wherein performing elution with the resultant binding beads further comprises:
introducing an eluate buffer from an eluate buffer chamber to the resultant binding beads in the mixing chamber;
mixing the resultant binding beads with the eluate buffer;
heating the mixing chamber to form an eluate; and
removing the eluate to an eluate collection chamber using centrifugal force.
